(19) United States
(12) Patent Application Publication
(10) Pub. No.: US 2020/0230253 A1
(43) Pub. Date: Jul. 23, 2020

(54) CONJUGATE OF IDURONATE-2-SULFATASE

(71) Applicant: HANMl PHARM. CO., LTD., Hwaseong-si, Gyeonggi-do (KR)

(72) Inventors: Dae Jin KIM, Hwaseong-si, Gyeonggi-do (KR); Jung Kuk KIM, Hwaseong-si, Gyeonggi-do (KR); Sung Youb JUNG, Hwaseong-si, Gyeonggi-do (KR); Se Chang KWON, Hwaseong-si, Gyeonggi-do (KR)

(73) Assignee: HANMl PHARM. CO., LTD., Hwaseong-si, Gyeonggi-do (KR)

(21) Appl. No.: 16/634,677

(22) PCT Filed: Jul. 27, 2018
(86) PCT No.: PCT/KR2018/008547

§ 371 Date: Jan. 28, 2020

(30) Foreign Application Priority Data
Jul. 28, 2017 (KR) 10-2017-0096470

(51) Int. Cl. A61K 47/68; A61K 47/60; A61K 38/46

(52) U.S. Cl. CPC A61K 47/68; A61K 47/60; C07K 2319/30; C12Y 301/06013; A61K 38/465

(57) ABSTRACT

Provided is a conjugate in which an immunoglobulin Fc region is linked to an iduronate-2-sulfatase enzyme through a non-peptide polymer linker moiety. Further, provided are a conjugate, a method for preparing the same, and a composition including the same in which a non-peptide polymer linker moiety is specifically linked to an immunoglobulin Fc.

### TECHNICAL FIELD

The present invention relates to a conjugate of a therapeutic enzyme in which an immunoglobulin Fc region is linked to the therapeutic enzyme through a non-peptide polymer linker moiety, a method for preparing the conjugate, and a composition including the same.

### BACKGROUND ART

Proteins such as therapeutic enzymes are easily denatured generally due to low stability and degraded by proteases in the blood, and are frequently administered to patients in order to maintain their blood concentration and potency. However, in the case of protein medicines that are mainly administered to patients in the form of injections, frequent injections to maintain the blood level of active polypeptides cause tremendous pain to the patient. In order to solve these problems, efforts have been made to increase the blood stability of therapeutic enzymes and to maintain the drug concentration in the blood for a long time to maximize the drug efficacy. The long-acting preparation of these therapeutic enzymes should not only increase the stability of therapeutic enzymes but also maintain sufficiently high potency of the drug itself and not cause an immune response in the patient.

In particular, Hunter syndrome (or Hunter disease), also called mucopolysaccharidosis II (MPS II), is a type of lysosomal storage disease (LSD). It is a fatal disease caused by a genetic defect of a specific enzyme and leads to death, and supplementary therapy of a defective enzyme is essential (Frances M. Platt et al., J Cell Biol. 2012 Nov. 26; (5): 723-34).

Enzyme supplementary therapy is a standard therapy in lysosomal storage diseases, and existing symptoms may be alleviated or disease progression may be lowered by supplementing deficient enzymes. However, the daily lives of patients and their families may be limited by the need to continuously administer the drug intravenously for 2 to 6 hours every 1 to 2 weeks.

In addition, a recombinant enzyme used in the treatment of Hunter syndrome has a very short duration due to a short half-life of 10 minutes to less than 3 hours in humans, causing inconvenience for patients who need to administer an enzyme during their lifetime, and as a result, it is very necessary to extend the half-life.

In addition, there is a problem in that the accumulation positions of the enzymes used in the treatment of Hunter syndrome are different from each other in vivo, and that the therapeutic enzyme does not reach the bone marrow, so that the development of a therapeutic agent for Hunter syndrome is becoming more and more necessary.

### DISCLOSURE

### Technical Problem

An object of the present invention is to provide an enzyme conjugate including iduronate-2-sulfatase.

Another object of the present invention is to provide a pharmaceutical composition for preventing or treating lysosomal storage diseases including the enzyme conjugate.

Yet another object of the present invention is to provide a method for preparing the enzyme conjugate.

### Technical Solution

An aspect of the present invention provides an enzyme conjugate in which an immunoglobulin Fc region is linked to iduronate-2-sulfatase through a non-peptide polymer linker moiety.

In a specific embodiment, the iduronate-2-sulfatase may treat mucopolysaccharidosis II (MPS II).

In another specific embodiment, the enzyme conjugate has increased transcytosis and bioavailability (BA) as compared with iduronate-2-sulfatase to which the immunoglobulin Fc region is not linked.

In yet another specific embodiment, the transcytosis is caused by binding of an immunoglobulin Fc region to a neonatal Fc receptor (FcRn).

In still another specific embodiment, the enzyme conjugate has increased tissue distribution as compared with iduronate-2-sulfatase to which the immunoglobulin Fc region is not linked.

In still yet another specific embodiment, the enzyme conjugate has increased bone marrow targeting as compared with iduronate-2-sulfatase to which the immunoglobulin Fc region is not linked.

In still yet another specific embodiment, the immunoglobulin Fc region is aglycosylated.

In still yet another specific embodiment, the immunoglobulin Fc region is constituted by 1 to 4 domains selected from the group consisting of CH1, CH2, CH3, and CH4 domains.

In still yet another specific embodiment, the immunoglobulin Fc region further includes a hinge region.

In still yet another specific embodiment, the immunoglobulin Fc region is an immunoglobulin Fc fragment derived from IgG, IgA, IgD, IgE, or IgM.

In still yet another specific embodiment, each domain of the immunoglobulin Fc region is a hybrid of domains having different origins derived from an immunoglobulin selected from the group consisting of IgG, IgA, IgD, IgE, and IgM.

In still yet another specific embodiment, the immunoglobulin Fc region is a dimer or multimer consisting of single-chain immunoglobulins composed of domains of the same origins.

In still yet another specific embodiment, the immunoglobulin Fc region is an IgG4 Fc fragment.

In still yet another specific embodiment, the immunoglobulin Fc region is a human aglycosylated IgG4 Fc fragment.

In still yet another specific embodiment, in the enzyme conjugate, a non-peptide polymer linker moiety is linked to a N-terminus of iduronate-2-sulfatase or a derivative thereof.

Another aspect of the present invention provides a pharmaceutical composition for preventing or treating mucopolysaccharidosis II (MPS II) including an enzyme conjugate of iduronate-2-sulfatase.

In a specific embodiment, the pharmaceutical composition increases transcytosis, bioavailability, tissue distribution, and bone marrow targeting.

Yet another aspect of the present invention provides a method for preparing an enzyme conjugate represented by the Chemical Formula 1, including: (a) reacting any one reactive functional group of a non-peptide polymer in which the same or different reactive functional groups are positioned to both termini with free iduronate-2-sulfatase to obtain a linked material in which a non-peptide polymer is covalently linked to the iduronate-2-sulfatase through a terminus; and

(b) reacting a reactive functional group of an unreacted terminus of the linked material with a biocompatible substance capable of increasing a half-life in vivo to covalently link the biocompatible substance with the linked material.

In one specific embodiment, the enzyme conjugate prepared by the method may be represented by Chemical Formula 1 below.

X—Lₐ—F [Chemical Formula 1]

Here, X is iduronate-2-sulfatase;

L is a non-peptide polymer linker;

a is 0 or a natural number, but when a is 2 or more, each L is independent of one another; and

F is a substance capable of increasing the in vivo half-life of X.

In one specific embodiment, the F may be selected from the group consisting of a polymer, a fatty acid, a cholesterol, albumin and fragments thereof, an albumin-binding substance, a polymer of repeat units of a specific amino acid sequence, an antibody, antibody fragments, an FcRn-binding substance, an in vivo connective tissue, a nucleotide, fibronectin, transferrin, saccharide, heparin, and elastin.

In a more specific embodiment, the FcRn-binding substance is an immunoglobulin Fc region.

In another specific embodiment, the iduronate-2-sulfatase may treat mucopolysaccharidosis II (MPS II).

In yet another specific embodiment, the non-peptide polymer is selected from the group consisting of polyethylene glycol, polypropylene glycol, an ethylene glycol-propylene glycol copolymer, polyoxyethylated polyol, polyvinyl alcohol, polysaccharide, dextran, polyvinyl ethyl ether, a biodegradable polymer, a lipid polymer, chitin, hyaluronic acid, and combinations thereof.

In still another specific embodiment, the non-peptide polymer is polyethylene glycol.

In still yet another specific embodiment, the reactive group of the non-peptide polymer of step (a) is selected from the group consisting of an aldehyde group, a maleimide group, and a succinimide derivative.

In still yet another specific embodiment, the aldehyde group is a propionaldehyde group or a butyraldehyde group.

In still yet another specific embodiment, the succinimide derivative is selected from the group consisting of succinimidyl carboxymethyl, succinimidyl valerate, succinimidyl methyl butanoate, succinimidyl methyl propionate, succinimidyl butanoate, succinimidyl propionate, N-hydroxysuccinimide, or succinimidyl carbonate.

In still yet another specific embodiment, the reactive functional groups are aldehyde groups at the both termini.

In still yet another specific embodiment, the non-peptide polymer has an aldehyde group and a maleimide group at each terminus as the reactive functional groups.

In still yet another specific embodiment, the non-peptide polymer has an aldehyde group and a succinimide group at each terminus as the reactive functional groups.

### Advantageous Effects

The enzyme conjugate of iduronate-2-sulfatase of the present invention is an enzyme conjugate having improved transcytosis, bioavailability, tissue distribution, and bone marrow targeting as well as improved duration of the enzyme in vivo, and the prepared enzyme conjugate may be usefully used for treatment of mucopolysaccharidosis II (MPS II).

### DESCRIPTION OF DRAWINGS

FIG. 1 is a diagram showing a PK experimental result of a long-acting conjugate of iduronate-2-sulfatase;

FIG. 2 is a diagram showing an in vitro enzyme activity of the long-acting conjugate of iduronate-2-sulfatase;

FIG. 3 is a diagram showing an in vitro intracellular absorption activity of the long-acting conjugate of iduronate-2-sulfatase; and

FIG. 4 is a diagram showing a result of comparing and verifying tissue distribution in ICR mice of the long-acting conjugate of the iduronate-2-sulfatase with that of iduronate-2-sulfatase without forming the long-acting conjugate (*p<0.05, **p<0.01, ***p<0.0005 vs. iduronate-2-sulfatase, unpaired t-test).

### DETAILED DESCRIPTION OF THE INVENTION

Detailed contents for implementing the present invention will be described below. Meanwhile, each description and exemplary embodiment described in the present application may be applied to other descriptions and exemplary embodiments. That is, all combinations of various elements described in the present application belong to the scope of the present invention. Further, the scope of the present invention may not be limited to the detailed description to be described below.

An aspect of the present invention to achieve the objects provides an enzyme conjugate in which an immunoglobulin Fc region is linked to iduronate-2-sulfatase through a non-peptide polymer linker moiety.

In one specific embodiment of the present invention, the enzyme conjugate is obtained by reacting a non-peptide polymer having reactive functional groups at both termini with a free Fc region and a free iduronate-2-sulfatase enzyme to form a covalent bond, and at this time, the non-peptide polymer is the same substance as the non-peptide polymer linker moiety in the type and number of repeating units and positions. The non-peptide polymer used in the reaction of free Fc region and free enzyme may be substances having terminal reactive functional groups having different chemical structures from the repeating unit at each end of the repeating units. In the process of preparing such an enzyme conjugate, a chemical reaction occurs with the free enzyme and the free immunoglobulin Fc region through the each terminal reactive functional group of the non-peptide polymer. The repeating units of the non-peptide polymer are linked to the enzyme and the Fc region due to the covalent bond generated when the terminal reactive functional groups are each converted by the chemical reaction to obtain the enzyme conjugate. That is, in the specific embodiment of the present invention, the non-peptide polymer is converted into the non-peptide polymer linker moiety in the enzyme conjugate through the preparing process.

The term “therapeutic enzyme” or “enzyme” of the present invention refers to an enzyme for treating diseases caused by lack, deficiency, dysfunction, or the like of the enzyme and refers to an enzyme for treating a subject having the diseases by enzyme replacement therapy, administration, or the like. Specifically, the therapeutic enzyme may be an enzyme for treating mucopolysaccharidosis II (MPS II) caused by lack or deficiency of a specific enzyme and more specifically, may be iduronate-2-sulfatase, but the present invention is not limited thereto.

The term “mucopolysaccharidosis (MPS)” of the present invention is also known as “mucopolysaccharosis”, which is a hereditary lyase deficiency of mucopolysaccharides. The mucopolysaccharidosis is a lysosomal storage disease caused by deficiency of a lysosome enzyme. Deficiency or the like of glycosidase, sulfatase, or acetyl transferase is known as a cause. The mucopolysaccharidosis is also referred to as gargoylism. The main symptom is excessive excretion of mucopolysaccharides in the urine. The mucopolysaccharidosis is currently classified into six disease types, in which type I includes Hurler syndrome and Scheie syndrome, type II includes Hunter syndrome, type III includes Sanfilippo syndrome types A, B, C, and D, type IV includes Morquio syndrome types A and B, type VI includes Maroteaux-Lamy syndrome, and type VII includes Sly syndrome.

The term “Hunter syndrome (Hunter disease)” of the present invention refers to a sex chromosomal recessive disorder caused by deficiency of iduronate-2-sulfatase (IDS), and it is known that heparan sulfate, and dermatan sulfate are accumulated due to the enzyme deficiency. Hunter syndrome has symptoms such as dysfunction, progressive hearing loss, pigmented retinal degeneration, papilledema, and hydrocephalus. In the present invention, the terms “mucopolysaccharidosis II” and “Hunter syndrome” may be used interchangeably.

The term “conjugate of iduronate-2-sulfatase” or “enzyme conjugate” of the present invention refers to the fact that iduronate-2-sulfatase, having an effect of treating Hunter syndrome, is linked to the immunoglobulin Fc region through the non-peptide polymer linker moiety to obtain an effect of preventing or treating Hunter syndrome by the enzyme.

The enzyme conjugate of the present invention may have an effect of increasing duration by linking a substance capable of increasing the half-life of the iduronate-2-sulfatase to the enzyme. Thus, the term “enzyme conjugate” of the present invention may be used interchangeably with “long-acting conjugate”.

In addition, the enzyme conjugate of the present invention may be used as a drug for enzymatic replacement therapy (ERT). The enzymatic replacement therapy can prevent or treat diseases through restoration of degraded enzyme functions by supplementing deficient or lacking enzymes which cause the diseases.

The conjugate including the iduronate-2-sulfatase of the present invention is administered to a patient with Hunter syndrome deficient in iduronate-2-sulfatase to alleviate or treat symptoms of the patient with Hunter syndrome. In addition, since the conjugate of the present invention has effects of increased duration and high transcytosis and bioavailability, an effect of preventing, improving, or treating Hunter syndrome may be exhibited with a small dose.

Specifically, the enzyme conjugate linked to the Fc region of the present invention has an effect whereby the Fc region binds to FcRn to increase the transcytosis of the Fc region compared to the enzyme having no Fc region linked thereto.

Although the enzyme is linked to the Fc region to form large molecules, the increased transcytosis mediated by FcRn may not only help maintain the intracellular uptake rate and perform a function of the enzyme itself because the therapeutic enzyme is absorbed into the body while maintaining the enzyme activity for a long period, and is thereby able to treat Hunter syndrome.

Meanwhile, the therapeutic enzyme, specifically, iduronate-2-sulfatase, which is used for the treatment of Hunter syndrome, has a disadvantage in that it requires intravenous injection due to low bioavailability (BA), and the intravenous injection causes inconvenience for the patient having to visit a hospital every time. In addition, such intravenous injection causes an allergic type hypersensitivity reaction, resulting in an infusion reaction related with drugs at a high frequency.

The enzyme conjugate of the present invention enhances bioavailability due to transcytosis caused by the FcRn binding site to enable subcutaneous injection rather than intravenous injection, and the change of the administration method enables self-injection to increase the convenience of the patient and decreases the infusion reaction to minimize the side effects.

In addition, the therapeutic enzymes, specifically, enzymes for the enzyme replacement therapy (ERT) used in Hunter syndrome, are required to remove waste products accumulated in specific tissues, so that even distribution to each tissue is very important. However, the currently used ERT enzymes are mainly distributed in the liver, and it is required to develop a drug capable of exhibiting distribution in various organs.

The enzyme conjugate including iduronate-2-sulfatase of the present invention has an increased half-life in the blood and excellent distribution having a high in vivo concentration in various tissues other than the liver, particularly, in bone marrow, spleen, and kidney due to transcytosis by the FcRn binding site, thereby maximizing therapeutic efficiency. In particular, the enzyme conjugate of the present invention exhibits high bone marrow targeting. The bone marrow tissue is a flexible structure within the bone and increases the targeting to the bone marrow tissue, and thus additional effects through pharmacological action in the bone marrow due to the ERT may be expected, thereby allowing effective treatment of Hunter syndrome.

In particular, the disease in the bone marrow causes a variety of side effects such as an abnormal symptom of bone or inhibition of bone formation, anemia through suppression of the production of erythrocyte-producing hormone and platelet-producing hormone, which are necessarily produced in the bone marrow, but in currently commercialized enzyme therapeutic agents, the above side effects frequently occur due to very low distribution of the bone marrow. In this respect, the enzyme conjugate including the iduronate-2-sulfatase of the present invention has high bone marrow targeting and may be used as a therapeutic agent for excellent enzyme replacement therapy.

Therefore, the enzyme conjugate of the present invention may have increased transcytosis, bioavailability, tissue distribution, and bone marrow targeting due to the binding of the immunoglobulin Fc region with the FcRn as compared with the enzyme to which the immunoglobulin Fc region does not bind.

The term “iduronate-2-sulfatase” of the present invention is a sulfatase enzyme related to Hunter syndrome (MPS-II), and an enzyme required for lysosomal lysis of heparan sulfate and dermatan sulfate. In one specific embodiment of the present invention, “idursulfase”, which is a form of the human iduronate-2-sulfatase, may be used as the iduronate-2-sulfatase. The idursulfase may be, for example, idursulfase alpha or idursulfase beta, but is not limited thereto.

The iduronate-2-sulfatase enzyme may be prepared or fabricated by a method known in the art. Specifically, the iduronate-2-sulfatase enzyme may be obtained by culturing animal cells into which an animal cell expression vector is inserted to be purified from a culture medium and purchasing commercially available enzymes, but the present invention is not limited thereto.

In one embodiment of the present invention, a conjugate in which an immunoglobulin Fc region and a non-peptide polymer are linked to iduronate-2-sulfatase was prepared (Example 3), and the in vitro and in vivo activities of the enzyme conjugate were confirmed (Examples 4 to 6).

As a result, it was confirmed that the iduronate-2-sulfatase of the enzyme conjugate increased all of the half-life, bioavailability, and tissue distribution while maintaining the enzyme activity despite binding with the Fc region.

The iduronate-2-sulfatase which may be included in the enzyme conjugate of the present invention may be in a natural form, and may be a natural type full length or a fragment composed of a part thereof. Alternatively, the iduronate-2-sulfatase may be an enzyme analog in which a variation selected from the group consisting of substitution, addition, deletion, modification, and combinations thereof occurs in some amino acids in a natural type sequence. The analog of the iduronate-2-sulfatase is not limited in the present invention as long as the analog has an activity that is higher than that of the natural type, and has an activity of at least a significant level as compared with the natural type.

The enzyme analogs may include the biosimilars and biobetters of the corresponding enzymes. For example, with respect to biosimilars, in consideration of the difference between a known enzyme and a host for its expression, the difference in glycosylation feature and the degree thereof, and the difference in the degree of substitution in a particular amino acid residue of the corresponding enzyme in light of the standard sequence where the degree of substitution is not 100% substitution, they belong to the biosimilar enzymes. The enzymes may be produced by genetic recombination in animal cells, E. coli, yeast, insect cells, plant cells, and in live animals, etc., and the preparation method is not limited thereto, and the enzymes may be commercially available enzymes.

Additionally, the enzymes may include an amino acid sequence which have a homology of at least 80%, more specifically 90%, and even more specifically 91%, 92%, 93%, 94%, 95%, 96%, 97%, 98%, or 99% or higher to that of the above enzymes or analogs thereof, and the enzymes may be obtained from microorganisms by recombinant technology or those which are commercially available, but are not limited thereto.

As used herein, the term “homology” represents the degree of similarity to the amino acid sequence of a wild-type protein or a nucleotide sequence encoding the amino acids, and it includes those sequences which have the same sequence at a percentage level described above to the amino acid sequences or nucleotide sequences of the present invention. The homology may be determined by comparing the two given sequences by the naked eye or may be determined using a bioinformatic algorithm, which enables the analysis of a homology by arranging the subject sequences for comparison. The homology between the two given amino acid sequences may be indicated as a percentage. The useful automated algorithm is available for use in GAP, BESTFIT, FASTA, and TFASTA computer software modules of Wisconsin Genetics Software Package (Genetics Computer Group, Madison, Wis., USA). The arrangement algorithm automated in the above modules includes sequence arrangement algorithms by Needleman & Wunsch, Pearson & Lipman, and Smith & Waterman. Other useful algorithms on sequence arrangement and homology determination are automated in software including FASTP, BLAST, BLAST2, PSIBLAST, and CLUSTAL W.

The amino acid sequences and nucleotide sequences encoding the same of the enzymes and analaogs thereof may be obtained from a known database such as the GenBank of NCBI, but are not limited thereto.

As used herein, the term “immunoglobulin Fc region” refers to a region of an immunoglobulin molecule, except for the variable regions of the heavy and light chains, the heavy-chain constant region 1 (C_{H}1) and the light-chain constant region 1 (C_{L}1) of an immunoglobulin. The immunoglobulin Fc region may further include a hinge region at the heavy-chain constant region. In particular, the immunoglobulin Fc region of the present invention may be a fragment including a part or entirety of the Fc region, and in the present invention, the immunoglobulin Fc region may be used interchangeably with an immunoglobulin fragment.

A native Fc has a sugar chain at position Asn297 of heavy-chain constant region 1, but E. coli-derived recombinant Fc is expressed as an aglycosylated form. The removal of sugar chains from Fc results in a decrease in binding affinity of Fc gamma receptors 1, 2, and 3 and complement (c1q) to heavy-chain constant region 1, leading to a decrease or loss in antibody-dependent cell-mediated cytotoxicity or complement-dependent cytotoxicity.

As used herein, the term “immunoglobulin constant region” may refer to an Fc fragment including heavy-chain constant region 2 (CH2) and heavy-chain constant region 3 (CH3) (or containing heavy-chain constant region 4 (CH4)), except for the variable regions of the heavy and light chains, the heavy-chain constant region 1 (CH1) and the light-chain constant region (CL) of an immunoglobulin, and may further include a hinge region at the heavy chain constant region. Further, the immunoglobulin constant region of the present invention may be an extended immunoglobulin constant region including a part or entirety of the Fc region including the heavy-chain constant region 1 (CH1) and/or the light-chain constant region (CL), except for the variable regions of the heavy and light chains of an immunoglobulin, as long as it has a physiological function substantially similar to or better than the native protein. Also, it may be a region having deletion in a relatively long portion of the amino acid sequence of CH2 and/or CH3. That is, the immunoglobulin constant region of the present invention may include (1) a CH1 domain, a CH2 domain, a CH3 domain, and a CH4 domain, (2) a CH1 domain and a CH2 domain, (3) a CH1 domain and a CH3 domain, (4) a CH2 domain and a CH3 domain, (5) a combination of one or more domains of the constant region and an immunoglobulin hinge region (or a portion of the hinge region), and (6) a dimer of each domain of the heavy-chain constant regions and the light-chain constant region. An immunoglobulin constant region including the immunoglobulin Fc fragment is a biodegradable polypeptide which can be metabolized in vivo and thus it can safely be used as a drug carrier. In addition, an immunoglobulin Fc fragment is more advantageous in terms of production, purification, and yield of a complex than an entire immunoglobulin molecule, owing to its relatively low molecular weight. Further, since it is devoid of Fab, which exhibits high non-homogeneity due to the difference in amino acid sequence from one antibody to another, it is expected to significantly enhance homogeneity and to reduce the possibility of inducing blood antigenicity.

Meanwhile, the immunoglobulin constant region may originate from humans or animals, such as cows, goats, pigs, mice, rabbits, hamsters, rats, guinea pigs, etc., and may preferably be of human origin. In addition, the immunoglobulin constant region may be selected from the group consisting of constant regions derived from IgG, IgA, IgD, IgE, IgM, or combinations or hybrids thereof, specifically, derived from IgG or IgM, which are the most abundant thereof in human blood, and most specifically, derived from IgG, which is known to improve the half-life of ligand-binding proteins. In the present invention, the immunoglobulin Fc region may be a dimer or multimer consisting of single-chain immunoglobulins composed of domains of the same origin.

As used herein, the term “combination” means that polypeptides encoding single chain immunoglobulin constant regions (specifically Fc regions) of the same origin are linked to a single-chain polypeptide of a different origin to form a dimer or multimer. That is, a dimer or a multimer may be prepared from two or more fragments selected from the group consisting of Fc fragments of IgG Fc, IgA Fc, IgM Fc, IgD Fc, and IgE Fc.

As used herein, the term “hybrid” means that sequences encoding two or more immunoglobulin constant regions of different origins are present in a single-chain of an immunoglobulin constant region (preferably, an Fc region). In the present invention, various hybrid forms are possible. That is, the hybrid domain may be composed of one to four domains selected from the group consisting of CH1, CH2, CH3, and CH4 of IgG Fc, IgM Fc, IgA Fc, IgE Fc, and IgD Fc, and may further include a hinge region. IgG may be divided into the IgG1, IgG2, IgG3, and IgG4 subclasses, and the present invention may include combinations or hybrids thereof, specifically, the IgG2 and IgG4 subclasses, and more specifically, the Fc region of IgG4 which rarely has effector functions such as complement dependent cytotoxicity (CDC).

The immunoglobulin constant region may have the glycosylated form to the same extent as, or to a greater or lesser extent than the native form or may be the deglycosylated form. Increased or decreased glycosylation or deglycosylation of the immunoglobulin constant region may be achieved by conventional methods, e.g., a chemical method, an enzymatic method, or a genetic engineering method using microorganisms. Herein, when deglycosylated, the complement (C1q) binding to an immunoglobulin constant region becomes significantly decreased and antibody-dependent cytotoxicity or complement-dependent cytotoxicity is reduced or removed, thereby not inducing unnecessary immune responses in vivo. In this regard, deglycosylated or aglycosylated immunoglobulin constant regions are more consistent with the purpose of drug carriers. Accordingly, the immunoglobulin Fc region may be even more specifically an aglycosylated Fc region derived from human IgG4, i.e., a human IgG4-derived aglycosylated Fc region. The human-derived Fc region is more preferable than a non-human derived Fc region, which may act as an antigen in the human body and cause undesirable immune responses such as the production of a new antibody against the antigen.

Further, the immunoglobulin constant region of the present invention includes not only the native amino acid sequence but also sequence derivatives (mutants) thereof. The amino acid sequence derivative means that it has an amino acid sequence different from the wild-type amino acid sequence as a result of deletion, insertion, conserved or non-conserved substitution of one or more amino acid residues, or a combination thereof. For example, amino acid residues at positions 214 to 238, 297 to 299, 318 to 322, or 327 to 331 in IgG Fc, known to be important for linkage, may be used as the sites suitable for modification. Various derivatives, such as those prepared by removing the sites capable of forming disulfide bonds, removing several N-terminal amino acids from native Fc, or adding methionine to the N-terminus of native Fc, may be used. In addition, complement fixation sites, e.g., C1q fixation sites or ADCC sites may be eliminated to remove the effector function. The techniques of preparing the sequence derivatives of the immunoglobulin constant region are disclosed in International Patent Publication Nos. WO 97/34631 and WO 96/32478, etc.

Amino acid substitutions in a protein or peptide molecule that do not alter the activity of a molecule are well known in the art (H. Neurath, R. L. Hill, The Proteins, Academic Press, New York, 1979). The most common substitutions occur between amino acid residues Ala/Ser, Val/Ile, Asp/Glu, Thr/Ser, Ala/Gly, Ala/Thr, Ser/Asn, Ala/Val, Ser/Gly, Thr/Phe, Ala/Pro, Lys/Arg, Asp/Asn, Leu/Ile, Leu/Val, Ala/Glu, and Asp/Gly, in both directions. Optionally, amino acids may be modified by phosphorylation, sulfation, acrylation, glycosylation, methylation, farnesylation, acetylation, amidation, etc.

The above-described immunoglobulin constant region derivative may be a derivative which has the same biological activity as that of the immunoglobulin constant region of the present invention, but has increased structural stability of the immunoglobulin constant region against heat, pH, etc. Further, the immunoglobulin constant region may be obtained from a native type isolated from humans or animals such as cows, goats, pigs, mice, rabbits, hamsters, rats, guinea pigs, etc., or may be their recombinants or derivatives obtained from transformed animal cells or microorganisms. Herein, they may be obtained from a native immunoglobulin by isolating whole immunoglobulins from human or animal organisms and treating them with a protease. Papain digests the native immunoglobulin into Fab and Fc regions, and pepsin treatment results in the production of pF'c and F(ab)2 fragments. These fragments may be subjected to size exclusion chromatography to isolate Fc or pF'c.

Preferably, a human-derived immunoglobulin constant region may be a recombinant immunoglobulin constant region that is obtained from a microorganism.

As used herein, the term “non-peptide polymer” includes a biocompatible polymer to which at least two repeating units are linked, and may be used interchangeably with “non-peptide linker”. The repeating units are linked together through a random covalent bond instead of a peptide bond. In the present invention, the non-peptide polymer may form a conjugate through a reaction with other element(s) which constitute(s) the conjugate, by including a reactive group(s) at an end thereof.

The term “non-peptide polymer linker moiety” of the present invention refers to a component in the conjugate formed by binding the non-peptide polymer having reactive functional groups at both termini to an immunoglobulin Fc region and an iduronate-2-sulfatase enzyme through each reactive group.

In one specific embodiment, the enzyme conjugate may include an immunoglobulin Fc region and an iduronate-2-sulfatase enzyme which are covalently linked to each other through a non-peptide polymer including reactive functional groups capable of binding to the immunoglobulin Fc region and the iduronate-2-sulfatase enzyme at both termini.

Specifically, although not particularly limited thereto, the non-peptide polymer may be one selected from the group consisting of polyethylene glycol, polypropylene glycol, an ethylene glycol-propylene glycol copolymer, polyoxyethylated polyol, polyvinyl alcohol, a polysaccharide, dextran, polyvinyl ethyl ether, a biodegradable polymer such as polylactic acid (PLA) and polylactic-glycolic acid (PLGA), a lipid polymer, chitins, hyaluronic acid, an oligonucleotide, and a combination thereof. In a more specific embodiment, the non-peptide polymer may be polyethylene glycol, but is not limited thereto. Additionally, the derivatives of the above materials already known in the art and the derivatives that can be easily produced at the technology level in the art also belong to the scope of the present invention.

The molecular weight of the non-peptide polymer may be in the range of exceeding 0 kDa to 200 kDa, and specifically, 1 kDa to 100 kDa, more specifically, 1 kDa to 50 kDa, even more specifically, 1 kDa to 20 kDa, even more specifically, 3.4 kDa to 10 kDa, even yet more specifically, about 3.4 kDa, but is not limited thereto.

The term “approximately” of the present invention is in a range including all of ±0.5, ±0.4, ±0.3, ±0.2, ±0.1, and the like, and includes all values which are equal to or similar to the values following the term of approximately, but the present invention is not limited thereto.

In one specific embodiment of the present invention, each terminus of the non-peptide polymer may have reactive functional groups. The reactive functional groups include a functional group of the immunoglobulin Fc region, such as an amine group or a thiol group, and a functional group of an iduronate-2-sulfatase enzyme, for example, a reactive functional group which may form a covalent bond by reacting with an amine group or a thiol group. These reactive functional groups may be the same as or different from each other at both ends.

More specifically, the non-peptide polymer may include a reactive group capable of binding to the immunoglobulin Fc and the iduronate-2-sulfatase enzyme at both termini, particularly, a reactive group capable of binding to an amine group located at the N-terminus or lysine, or a thiol group of cysteine of the iduronate-2-sulfatase enzyme or the immunoglobulin Fc region, but the present invention is not limited thereto.

More specifically, the reactive functional group of the non-peptide polymer may be selected from the group consisting of an aldehyde group, a maleimide group, and a succinimide derivative, but is not limited thereto.

Hereinabove, the aldehyde group may include, for example, a propionaldehyde group or a butyraldehyde group, but is not limited thereto.

As an example, the non-peptide polymer used in one embodiment of the present invention has a polyethylene glycol backbone as a linear polymer having aldehyde (-CHO) groups at both termini. The size of the non-peptide polymer is determined by the number of repeating units constituting the backbone, and the aldehyde as the reactive group of the non-peptide polymer may covalently bind to the amine group of the enzyme, specifically, —NH₂ of a lysine residue or —NH₂ of a N-terminus. The aldehyde group of the non-peptide polymer reacts specifically with —NH₂ of the N-terminus of the enzyme under a specific reaction condition, and a non-peptide polymer linked material covalently binding to the enzyme may be obtained through purification after the reaction.

Hereinabove, as the succinimide derivative, succinimidyl carboxymethyl, succinimidyl valerate, succinimidyl methyl butanoate, succinimidyl methyl propionate, succinimidyl butanoate, succinimidyl propionate, N-hydroxysuccinimide, or succinimidyl carbonate may be used, but it is not limited thereto.

The non-peptide polymer may be linked to the immunoglobulin Fc and the iduronate-2-sulfatase enzyme through the reactive groups to be converted to a non-peptide polymer linker moiety.

Further, a final product generated by reductive amination via an aldehyde bond is much more stable than the final product linked by an amide bond. The aldehyde reactive group selectively reacts with the N-terminus at a low pH and may form a covalent bond with a lysine residue at a high pH, for example, pH 9.0.

The reactive functional groups at the termini of the non-peptide polymer of the present invention may be the same as or different from each other. The non-peptide polymer may have an aldehyde group as a reactive functional group at both termini, and the non-peptide polymer may have an aldehyde group and a maleimide group at both termini as reactive functional groups, or may have an aldehyde group and a succinimide group at both termini as reactive functional groups, but the present invention is not limited thereto.

For example, the non-peptide polymer may have a maleimide group at one terminus and an aldehyde group, a propionaldehyde group, or a butyraldehyde group at the other terminus as each of the reactive functional groups. As another example, the non-peptide polymer may have a succinimidyl group at one terminus and a propionaldehyde group or butyraldehyde group at the other terminus.

When poly(ethylene glycol) having a hydroxyl group at a propion-side terminus is used as the non-peptide polymer, the enzyme conjugates of the present invention may be prepared by activating the hydroxy group into various reactive functional groups by a known chemical reaction or using the poly(ethylene glycol) having a commercially-obtainable modified reactive functional group.

In one specific embodiment, the reactive functional group of the non-peptide polymer may be linked to a cysteine residue, particularly, a —SH group of iduronate-2-sulfatase, but is not limited thereto.

In the case of using maleimide-PEG-aldehyde, the maleimide group may be linked to a —SH group of the iduronate-2-sulfatase enzyme by a thioether bond and the aldehyde group may be linked with a —NH₂ group of the immunoglobulin Fc through a reductive amination reaction, but the present invention is not limited thereto, and this corresponds to one example.

In one specific embodiment of the present invention in which polyethylene glycol having an aldehyde reactive functional group at one terminus is used as a non-peptide polymer, a linker functional group having a structure of —CH₂CH₂— at an oxygen atom located the end of the terminus of the PEG forms the non-peptide polymer linker moiety through the reductive amination. That is, in the enzyme conjugate of this specific embodiment, the iduronate-2-sulfatase is linked to the immunoglobulin Fc through the non-peptide polymer linker moiety having a chemical structure such as -PEG-CH2NH— or NHCH₂CH₂CH₂-PEG-.

In another specific embodiment of the present invention in which polyethylene glycol having a maleimide-reactive functional group at one terminus is used as the non-peptide polymer, the maleimide-side terminus is linked to a sulfur atom to a cysteine (for example, cysteine of the iduronate-2-sulfatase) through the thioether bond to form the non-peptide polymer linker moiety. The thioether bond described above may include a structure of

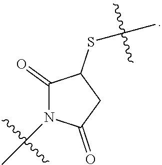 [SMILES: *SC1CC(=O)N(*)C1=O]

However, the present invention is not particularly limited to the above-described examples, and this corresponds to one example. Also, in the conjugate, the reactive functional group of the non-peptide polymer may be linked to —NH₂ located at the N-terminus of the immunoglobulin Fc region or the iduronate-2-sulfatase, which corresponds to one example. Specifically, the iduronate-2-sulfatase of the present invention may be linked to the non-peptide polymer having a reactive functional group through the N-terminus, but is not limited thereto.

The term “N-terminus” of the present invention refers to an amino terminus of the peptide and refers to a position capable of binding with the non-peptide polymer for the purpose of the present invention. As an example, the present invention is not limited thereto, but the N-terminus may include both an amino acid residue at the very end of the N-terminus and an amino acid residue around the N-terminus, and particularly, may include first to 20th amino acid residues from the very end.

Also, in the conjugate, the reactive functional group of the non-peptide polymer may be linked to an amine group of another residue other than the N-terminus of the immunoglobulin Fc region or the iduronate-2-sulfatase, but this corresponds to one example. Specifically, the residue may be a lysine residue of the iduronate-2-sulfatase or the immunoglobulin Fc region, but is not limited thereto.

That is, in the enzyme conjugate of the present invention, the reactive functional group of the non-peptide polymer is covalently linked with the amine group of the iduronate-2-sulfatase or the immunoglobulin Fc region, but the present invention is not limited thereto.

The enzyme conjugate of the present invention may exhibit a difference in activity depending on a binding position of the non-peptide polymer. That is, the half-life or activity in vivo may be improved by position-specific binding between the non-peptide polymer and the enzyme.

Another aspect of the present invention provides a pharmaceutical composition for preventing or treating mucopolysaccharidosis II (MPS II) including a conjugate of iduronate-2-sulfatase.

In the conjugate of iduronate-2-sulfatase, iduronate-2-sulfatase and an immunoglobulin Fc region may be linked to each other through a non-peptide polymer linker moiety.

The terms “immunoglobulin Fc region”, “non-peptide polymer linker moiety”, “enzyme conjugate”, and “mucopolysaccharidosis II” of the present invention are as described above.

The term “prevention” of the present invention means all actions that inhibit or delay the onset of the disease by administering an iduronate-2-sulfatase enzyme or a composition including the iduronate-2-sulfatase enzyme for the treatment of the mucopolysaccharidosis II or Hunter syndrome. The term “treatment” means all actions in which the symptoms of the mucopolysaccharidosis II or Hunter syndrome are alleviated or advantageous by administering the enzyme or the composition including the enzyme.

In the present invention, the term “administration” means introduction of a predetermined substance into a patient by a certain suitable method. The administration route of the composition is not particularly limited thereto, but the composition may be administered through any general route which may reach an in vivo target. For example, the general route may include intraperitoneal, intravenous, intramuscular, subcutaneous, intradermal, oral, topical, intranasal, intrapulmonary, or intrarectal administration.

The pharmaceutical composition for preventing or treating the mucopolysaccharidosis II of the present invention may have an effect of treating the mucopolysaccharidosis II by administering a deficient or lacking enzyme causing the mucopolysaccharidosis II to a subject having the disease to restore a function of the deficient or lacking enzyme.

A specific embodiment of the present invention provides a pharmaceutical composition that increases transcytosis, bioavailability, tissue distribution, and bone marrow targeting.

In the pharmaceutical composition of the present invention, the enzyme conjugate in which the iduronate-2-sulfatase is linked to the Fc region may easily pass through a cell membrane through an intrinsic bond with the FcRn receptor and also more effectively reach tissues in the blood vessel.

Thus, the pharmaceutical composition may have increased effects of transcytosis, bioavailability, tissue distribution, and bone marrow targeting, and exhibit an excellent therapeutic effect of the mucopolysaccharidosis II.

The pharmaceutical composition of the present invention may further contain a pharmaceutically acceptable carrier, excipient, or diluent. The pharmaceutically acceptable carrier, excipient, or diluent may be non-naturally occurring. The pharmaceutically acceptable carrier may include, for oral administration, a binder, a glidant, a disintegrant, an excipient, a solubilizing agent, a dispersant, a stabilizing agent, a suspending agent, a coloring agent, a flavoring agent, etc.; for injections, a buffering agent, a preserving agent, an analgesic, a solubilizing agent, an isotonic agent, a stabilizing agent, etc., which may be combined to be used; and for topical administrations, a base, an excipient, a lubricant, a preserving agent, etc., but is not limited thereto.

As used herein, the term “pharmaceutically acceptable” refers to the properties of having a sufficient amount to exhibit a therapeutic effect and not causing adverse effects, and may be easily determined by a skilled person in the art based on the factors well-known in the medical field, such as the kind of disease, age, body weight, health status, sex, drug sensitivity of a patient, administration route, administration method, administration frequency, duration of treatment, a drug(s) to be mixed or administered simultaneously, etc.

The formulation type of the composition of the present invention may be prepared variously by combining with a pharmaceutically acceptable carrier described above. For example, for oral administration, the composition may be formulated into tablets, troches, capsules, elixirs, suspensions, syrups, wafers, etc. For injections, the composition may be formulated into unit-dose ampoules or multi-dose containers. The composition may also be formulated into solutions, suspensions, tablets, capsules, sustained-release formulations, etc.

Meanwhile, examples of suitable carriers, excipients, and diluents may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, etc. Additionally, the composition may further contain a filler, an anti-coagulant, a lubricant, a humectant, a flavoring agent, a preservative, etc.

Additionally, the pharmaceutical composition of the present invention may be prepared in any formulation type selected from the group consisting of tablets, pills, powders, granules, capsules, suspensions, liquid medicine for internal use, emulsions, syrups, sterile aqueous solutions, non-aqueous solvents, lyophilized formulations, and suppositories.

Additionally, the composition may be formulated into a unit dosage form suitable for the patient's body, and is specifically formulated into a preparation useful for protein drugs according to the typical method in the pharmaceutical field so as to be administered by an oral or parenteral route, such as intradermally, intravenously, intramuscularly, intraarterially, intramedullarily, intrathecally, intraventricularly, pulmonarily, transdermally, subcutaneously, intraperitoneally, intranasally, intragastrically, topically, sublingually, vaginally, or rectally, but the administration route is not limited thereto.

Additionally, the conjugate may be used by mixing with various pharmaceutically acceptable carriers approved as pharmaceutical drugs such as physiological saline or organic solvents. For increasing stability or absorptivity, carbohydrates such as glucose, sucrose, or dextrans, antioxidants such as ascorbic acid or glutathione, chelating agents, low molecular weight proteins, or other stabilizers may be used as pharmaceutical drugs.

The administration dose and frequency of the pharmaceutical composition of the present invention are determined by the type of active ingredient(s) together with various factors, such as the disease to be treated, administration route, patient's age, gender, and body weight, and severity of the disease.

The total effective dose of the composition of the present invention may be administered to a patient in a single dose or may be administered for a long period of time in multiple doses according to a fractionated treatment protocol. In the pharmaceutical composition of the present invention, the content of the active ingredient(s) may vary depending on the disease severity. Specifically, the total daily dose of the conjugate of the present invention may be about 0.0001 mg to 500 mg per 1 kg of body weight of a patient. However, the effective dose of the conjugate is determined considering various factors including patient's age, body weight, health conditions, gender, disease severity, diet, and excretion rate, in addition to administration route and treatment frequency of the pharmaceutical composition. In this regard, those skilled in the art may easily determine the effective dose suitable for the particular use of the pharmaceutical composition of the present invention. The pharmaceutical composition according to the present invention is not particularly limited to the formulation and administration route and mode, as long as it shows the effects of the present invention.

Yet another aspect of the present invention provides a method for preventing or treating mucopolysaccharidosis II (MPS II) including administering a pharmaceutical composition including an enzyme conjugate of iduronate-2-sulfatase to a subject in need thereof.

The enzyme conjugate, the composition including the same, the mucopolysaccharidosis II (MPS II), prevention, and treatment are as described above.

The term “subject” of the present invention is a subject suspected of having mucopolysaccharidosis II, and the subject refers to mammals such as rats and livestock including the humans in which the corresponding disease is developed or to be developed, but includes subjects which can be treated by the enzyme conjugate or the composition including the same of the present invention without limitation.

The method of the present invention may include administering a pharmaceutical composition including the enzyme conjugate in a pharmaceutically effective amount. The suitable total daily dose may be determined by a physician within accurate medical judgment, and administered once or several times. However, with respect to the objects of the present invention, the specific therapeutically effective dose for a specific patient may vary depending on various factors including the kind and degree of the response to be achieved, concrete compositions according to whether other agents are used therewith or not, the patient's age, body weight, health condition, gender, and diet, administration time, administration route, the secretion rate of the composition, the time period of therapy, other drugs used in combination or coincident with the particular composition, and like factors well known in the medical arts.

Still another aspect of the present invention provides a composition including the enzyme conjugate for increasing transcytosis, bioavailability, tissue distribution, and bone marrow targeting of the enzyme.

Particularly, the enzyme conjugate may be an enzyme conjugate in which iduronate-2-sulfatase and an immunoglobulin Fc region are linked to each other through a non-peptide polymer linker moiety, but is not limited thereto.

Still yet another aspect of the present invention provides a method for increasing transcytosis, bioavailability, tissue distribution, and bone marrow targeting of an enzyme, including administering the enzyme conjugate or a composition including the enzyme conjugate to a subject in need thereof.

Particularly, the enzyme conjugate may be an enzyme conjugate in which iduronate-2-sulfatase and an immunoglobulin Fc region are linked to each other through a non-peptide polymer linker moiety, but is not limited thereto.

Still yet another aspect of the present invention provides a use for preventing or treating mucopolysaccharidosis II (MPS II) of the enzyme conjugate.

Particularly, the enzyme conjugate may be an enzyme conjugate in which iduronate-2-sulfatase and an immunoglobulin Fc region are linked to each other through a non-peptide polymer linker moiety, but is not limited thereto.

Still yet another aspect of the present invention provides a use for increasing transcytosis, bioavailability, tissue distribution, and bone marrow targeting of an enzyme of the enzyme conjugate or the composition including the enzyme conjugate.

Particularly, the enzyme conjugate may be an enzyme conjugate in which iduronate-2-sulfatase and an immunoglobulin Fc region are linked to each other through a non-peptide polymer linker moiety.

Still yet another aspect of the present invention provides a method for preparing an enzyme conjugate represented by the Chemical Formula 1 including:

(a) reacting any one reactive functional group of a non-peptide polymer in which the same or different reactive functional groups are positioned at both termini with free iduronate-2-sulfatase to obtain a linked material in which a non-peptide polymer is covalently linked to the iduronate-2-sulfatase through the terminus; and

(b) reacting a reactive functional group of an unreacted terminus of the linked material with a biocompatible substance capable of increasing a half-life in vivo to covalently link the biocompatible substance with the linked material.

X—Lₐ—F [Chemical Formula 1]

Here, X is iduronate-2-sulfatase;

L is a non-peptide polymer linker moiety;

a is 0 or a natural number, but when a is 2 or more, each L is independent of one another; and

F is a substance capable of increasing the in vivo half-life of X.

In the present invention, the F may be selected from the group consisting of a polymer, a fatty acid, a cholesterol, albumin and fragments thereof, an albumin binding substance, a polymer of repeat units of a specific amino acid sequence, an antibody, antibody fragments, an FcRn binding substance, an in vivo connective tissue, a nucleotide, fibronectin, transferrin, saccharide, heparin, and elastin. More specifically, the FcRn binding substance may be an immunoglobulin Fc region, but the present invention is not limited thereto.

The F may be linked to the X by a covalent chemical bond or a non-covalent chemical bond, and the F and the X may be linked to each other through the L by a covalent chemical bond, a non-covalent chemical bond, or a combination thereof.

Particularly, the L may be a peptide polymer or a non-peptide polymer.

When the L is the peptide polymer, the L may include one or more amino acids, and for example, 1 to 1000 amino acids, but the present invention is not particularly limited thereto. In the present invention, various peptide linkers known for linking the F and the X may be used, and for example, a [GS]ₓ linker, a [GGGS]ₓ linker, a [GGGGS]ₓ linker, and the like are included, and herein, x may be a natural number of 1 or more. However, the present invention is not limited to the examples.

When the L is the non-peptide polymer, the non-peptide polymer may be selected from the group consisting of polyethylene glycol, polypropylene glycol, a copolymer of ethylene glycol and propylene glycol, polyoxyethylated polyol, polyvinyl alcohol, polysaccharides, dextran, polyvinyl ethyl ether, biodegradable polymers, lipopolymers, chitins, hyaluronic acid, and combinations thereof, and more particularly, polyethylene glycol, but the present invention is not particularly limited thereto.

In the present invention, a may be 0 or a natural number, and when a is 0, the enzyme conjugate of the present invention does not include a non-peptide polymer, and may be a conjugate in which the iduronate-2-sulfatase is linked to the immunoglobulin Fc region through a peptide linkage (fusion).

Further, the reactive functional group may be selected from the group consisting of an aldehyde group, a maleimide group, and a succinimide derivative. More specifically, the aldehyde group may be a propionaldehyde group or a butyraldehyde group, or the succinimide derivative may be succinimidyl carboxymethyl, succinimidyl valerate, succinimidyl methyl butanoate, succinimidyl methyl propionate, succinimidyl butanoate, succinimidyl propionate, N-hydroxysuccinimide, or succinimidyl carbonate, but the present invention is not limited thereto.

The non-peptide polymer may have aldehyde groups at both termini as reactive functional groups, have an aldehyde group and a maleimide group at each terminus as reactive functional groups, or have an aldehyde group and a succinimide group at each terminus as reactive functional groups, but the present invention is not limited thereto.

A method for preparing the enzyme conjugate of the present invention may include preparing a linked material in which iduronate-2-sulfatase and the non-peptide polymer covalently bind to each other; and preparing and obtaining a conjugate by linking the linked material with the immunoglobulin Fc region, but the present invention is not limited thereto.

The term “linked material” of the present invention refers to an intermediate of covalently binding the non-peptide polymer to the iduronate-2-sulfatase, and the conjugate may be prepared by then binding a reactive functional group of an unreacted terminus of the non-peptide polymer of the linked material to the immunoglobulin Fc region, but the present invention is not limited thereto.

Further, a specific embodiment of the present invention provides a method for preparing an enzyme conjugate, which further includes isolating the conjugate in which the non-peptide polymer linker moiety is linked to the N-terminus of the iduronate-2-sulfatase enzyme.

In the enzyme conjugate prepared by the preparation method of the present invention, since the iduronate-2-sulfatase is linked to the Fc region, the enzyme conjugate may easily pass through a cell membrane through an intrinsic bond with the FcRn receptor of the Fc region and also more effectively reach tissues in the blood vessel.

All of the increased transcytosis, bioavailability, and tissue distribution of the iduronate-2-sulfatase enzyme, contained in the enzyme conjugate, can help the enzyme to exhibit effective therapeutic effects of the Hunter syndrome while maintaining the enzyme activities in vivo.

In addition, the enzyme conjugate of the present invention may increase the half-life of the enzyme or the derivative (analog) thereof.

Hereinafter, the present invention will be described in more detail through Examples. Examples are to describe the present invention in detail, and the scope of the present invention is not limited to Examples.

### Example 1: Preparation of iduronate-2-sulfatase Enzyme

In order to prepare an enzyme conjugate of iduronate-2-sulfatase of the present invention, the following processes were performed. First, the iduronate-2-sulfatase used Elaprase manufactured by Shire.

### Example 2: Preparation of Enzyme Conjugate—1

An iduronate-2-sulfatase linked material in which a non-peptide polymer was linked to an amine group of the iduronate-2-sulfatase enzyme of Example 1 by a covalent bond was prepared and purified.

In order to conjugate the 10 kDa aldehyde-polyethylene glycol-aldehyde (NOF (Japan), M12N535, 10 kDa, ALD-PEG-ALD) linker to an amine group of the iduronate-2-sulfatase, the iduronate-2-sulfatase and the 10 kDa ALD-PEG-ALD were reacted with each other at a molar ratio of 1:20 with a concentration of the iduronate-2-sulfatase of 10 mg/mL at 4° C. to 8° C. for about 2 hours. At this time, the reaction was performed in 100 mM potassium phosphate at pH 6.0, and 20 mM sodium cyanoborohydride was added thereto as a reducing agent. The reaction solution was obtained by purifying unreacted iduronate-2-sulfatase from a mono-bound iduronate-2-sulfatase linked material using a Source 15Q (from GE in USA) column using a buffer containing 10 mM sodium phosphate at pH 6.0 and a sodium chloride concentration gradient.

### Example 3: Preparation of Enzyme Conjugate—2

The immunoglobulin Fc region was conjugated to the linked material of the non-peptide polymer and the iduronate-2-sulfatase purified in Example 2.

The purified iduronate-2-sulfatase linked material and the immunoglobulin Fc fragment were reacted with each other at a molar ratio of 1:10 and the whole protein concentration of 70 mg/mL to 75 mg/mL at 4° C. to 8° C. for about 16 hours. At this time, the reaction solution was 100 mM potassium phosphate at pH 6.0, and 20 mM sodium cyanoborohydride was added as a reducing agent. After the reaction was completed, the reaction solution was applied to a Source 15Q (from GE in USA) column using a buffer containing 10 mM sodium phosphate at pH 6.0 and a sodium chloride concentration gradient, a Source 15Iso (from GE in USA) column using a 20 mM sodium phosphate at pH 6.0 and an ammonium sulfate concentration gradient, and finally a Protein A (from GE in USA) using 20 mM Tris at pH 7.5, 5% (v/v) glycerol and a concentration gradient of 100 mM citric acid monohydrate at pH 3.7, 150 mM sodium chloride and 10% glycerol buffer (GE) to purify the conjugate in which the immunoglobulin Fc was linked to the iduronate-2-sulfatase by a covalent bond by a polyethylene glycol linker.

In Example 3, the finally prepared iduronate-2-sulfatase conjugate had a form in which the N-terminus of an iduronate-2-sulfatase monomer is linked to one side chain (N-terminal proline) of the immunoglobulin Fc having two chains via the polyethylene glycol linker.

### Example 4: Pharmacokinetic Experiment of Long-Acting iduronate-2-sulfatase Conjugate

The present inventors investigated the pharmacokinetics of the iduronate-2-sulfatase enzyme conjugate prepared in the above Examples to confirm the effect of the conjugate.

For this purpose, three ICR mice per blood sampling point of each group were administered with iduronate-2-sulfatase (idursulfase, control group) and the long-acting iduronate-2-sulfatase conjugate prepared in the above Example, and then stability in the blood and pharmacokinetic parameters were compared.

Specifically, the control group was intravenously injected with 1 mg/kg of iduronate-2-sulfatase, and then the blood was collected after 0, 0.25, 0.5, 1, 2, 4, 8, 24, 48, 72, 96, 120, 144, and 168 hours, and the experimental group was subcutaneously injected with 1 mg/kg each of the long-acting iduronate-2-sulfatase conjugate based on the iduronate-2-sulfatase, and then the blood was collected after 0, 1, 2, 4, 8, 24, 48, 72, 96, 120, 144, and 168 hours. A serum protein amount was measured by an ELISA method using an antibody against iduronate-2-sulfatase.

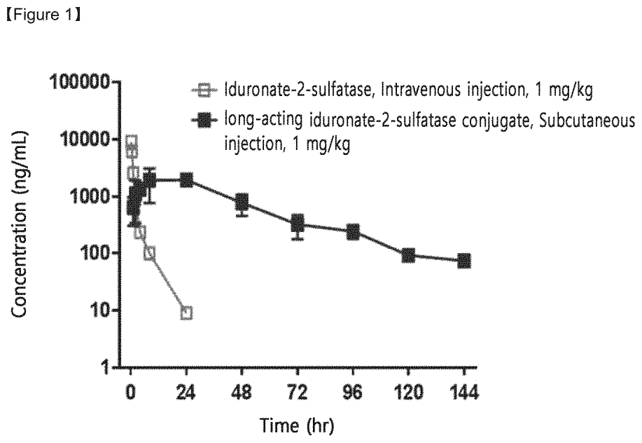

The pharmacokinetic analysis results were shown in FIG. 1.

Specifically, it was confirmed that both the half-life in the blood and the area under curve (AUC) for the drug molecule were increased in the case of the long-acting iduronate-2-sulfatase conjugate as compared with the control group. In particular, it was confirmed that the half-life in the blood of iduronate-2-sulfatase was 4.5 hours, whereas the half-life in the blood of the long-acting iduronate-2-sulfatase conjugate was 30.6 hours, which was about 7 times better than that of the iduronate-2-sulfatase, and the AUC was also increased about 9 times or more.

The present inventors attempted to confirm the in vitro activity of each enzyme conjugate as follows.

### Example 5: Confirmation of Activity of iduronate-2-sulfatase Enzyme Conjugate

### Example 5-1: In Vitro Enzyme Activity of Long-Acting iduronate-2-sulfatase Conjugate

The present inventors conducted measurement of in vitro enzyme activity to measure an enzyme activity change by the preparation of the long-acting iduronate-2-sulfatase conjugate.

Specifically, 4-methylumbelliferyl α-L-idopyranosiduronic acid-2-sulfate sodium salt (4MU-α-IdopyraA-2), known as an enzyme substrate, was reacted with iduronate-2-sulfatase and an long-acting iduronate-2-sulfatase conjugate at 37° C. for 4 hours and then reacted again with a secondary reactive enzyme, α-iduronidase, at 37° C. for 24 hours. The enzyme activity of the corresponding substance was measured by measuring the fluorescence of the 4-methylumbelliferone (4MU) ultimately produced.

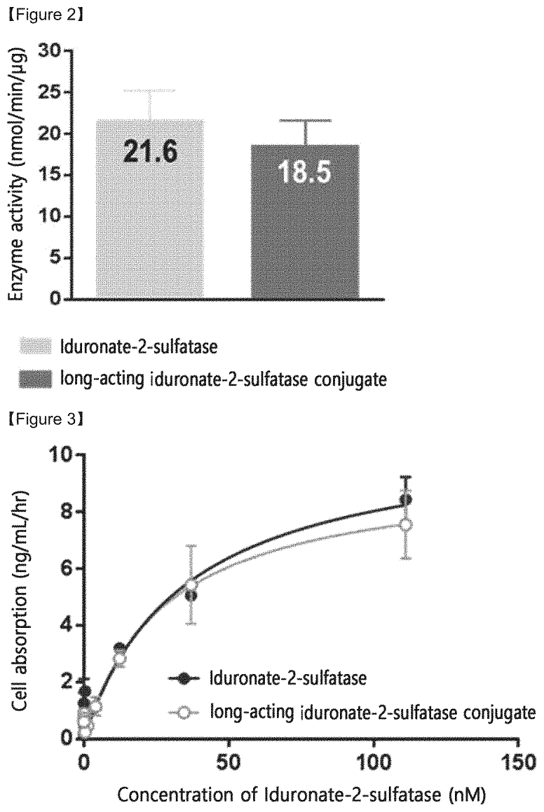

As a result, it was confirmed that enzyme activities of the iduronate-2-sulfatase and the long-acting iduronate-2-sulfatase conjugate were 21.6±3.63 nmol/min/pg and 18.5±3.12 nmol/min/pg, respectively. In conclusion, the enzyme activity of the long-acting iduronate-2-sulfatase conjugate was 85.7% compared to the iduronate-2-sulfatase, and this decrease in enzyme activity may be compensated by improved PK due to conjugation of the long-acting conjugate (FIG. 2).

### Example 5-2: Intracellular Absorption Activity of Long-Acting iduronate-2-sulfatase Conjugate

Since the iduronate-2-sulfatase acts after being absorbed into cells via a mannose phosphate 6 receptor (M6PR), whether or not the preparation of the long-acting iduronate-2-sulfatase conjugate affects the intracellular absorption activity of the enzyme was confirmed as follows.

First, HepG2 cell (human hepatocarcinoma), which was known to express M6PR, was treated with each of the iduronate-2-sulfatase and the long-acting iduronate-2-sulfatase conjugate, and then intracellular absorption was induced at 37° C. After 24 hours, the iduronate-2-sulfatase and the long-acting iduronate-2-sulfatase conjugate present in the cells were analyzed by ELISA.

As a result, the intracellular absorption activity (Vₘₐₓ=10.83 ng/mL) of the long-acting iduronate-2-sulfatase conjugate was about 81.4% compared with the intracellular absorption activity (Vₘₐₓ=9.50 nM) of the iduronate-2-sulfatase. This decrease in M6PR binding and intracellular absorption activity (vs. iduronate-2-sulfatase) may be compensated by improved PK due to conjugation of the long-acting conjugate due to the steric hindrance effect of the long-acting conjugate (FIG. 3).

### Example 5-3: Confirmation of Binding Affinity of Long-Acting iduronate-2-sulfatase Conjugate to M6PR

To confirm the binding affinity to a mannose phosphate 6 receptor (M6PR) of the iduronate-2-sulfatase and the long-acting iduronate-2-sulfatase conjugate, the following analysis was performed using surface plasmon resonance (BIACORE T200, GE Healthcare).

Specifically, the M6PR was immobilized on a CM5 chip by amine coupling. Then, the iduronate-2-sulfatase was diluted to 200 nM to 12.5 nM with an HBS-EP buffer, conjugated to the chip on which the M6PR was immobilized for 10 minutes, and dissociated for 6 minutes, and the binding affinity was calculated by BIAevaluation software. The relative binding affinity of the long-acting iduronate-2-sulfatase conjugate was quantified as compared with the iduronate-2-sulfatase.

As a result, it was confirmed that the long-acting iduronate-2-sulfatase conjugate had a receptor binding affinity of about 88% as compared to the iduronate-2-sulfatase (Table 1). It appears that the relatively low binding affinity of the long-acting iduronate-2-sulfatase conjugate to M6PR was due to the same reason that a physiologically active polypeptide conventionally shows a decrease in its binding affinity to its receptors when it forms a fusion protein with an Fc region. While it is not intended to be bound by any particular theory of the principles of operation of the present invention, but merely for the purpose of facilitating the understanding of the invention, when the immunoglobulin Fc and iduronate-2-sulfatase were covalently bonded, it was shown that the iduronate-2-sulfatase site received steric hindrance and the binding force to the receptor decreased.

TABLE 1

| Substance name | ka (1/Ms, ×10⁵) | kd (1/s, ×10⁻³) | KD (nM) |
|---|---|---|---|
| iduronate-2-sulfatase | 1.36 ± 0.03 | 3.95 ± 0.37 | 29.14 ± 3.39 |
| | (100%) | (100%) | (100%) |
| long-acting | 0.92 ± 0.08 | 3.07 ± 0.76 | 33.15 ± 5.45 |
| iduronate-2-sulfatase | (67%) | (78%) | (88%) |
| conjugate | | | |

The results of confirming the activity of the long-acting iduronate-2-sulfatase conjugate confirmed in the above Examples are summarized in Table 2 below.

TABLE 2

| In vitro activity (vs. 1st ERT) | | | In vivo activity (vs. 1st ERT) | | |
|---|---|---|---|---|---|
| | M6PR | | | | |
| Enzyme | binding | Cell | PK | | Tissue |
| activity | affinity | absorption | t½ | AUC | distribution |
| 86% | 88% | 81% | 6.8 times | 9.1 times | High target |
| | | | | | tissue |
| | | | | | distribution |
| | | | | | (kidney, liver, |
| | | | | | spleen, |
| | | | | | heart, bone |
| | | | | | marrow) |

### Example 6: Confirmation of Tissue Distribution of Long-Acting iduronate-2-sulfatase Conjugate

In three ICR mice per each blood sampling point of each group, tissue and organ distributions of iduronate-2-sulfatase (control group) and the long-acting iduronate-2-sulfatase conjugate prepared above were compared.

Specifically, the control group was intravenously injected with 1.0 mg/kg of iduronate-2-sulfatase and then organs were extracted at 1, 8, 48, and 144 hours. In the experimental group, the long-acting iduronate-2-sulfatase conjugate was subcutaneously injected at 1.0 mg/kg each on the basis of iduronate-2-sulfatase, and then the organs were extracted at 1, 8, 48, and 144 hours, and thereafter, respective substance concentrations in tissues (serum, kidney, liver, spleen, heart, bone marrow, and lung) were measured and compared by an ELISA method.

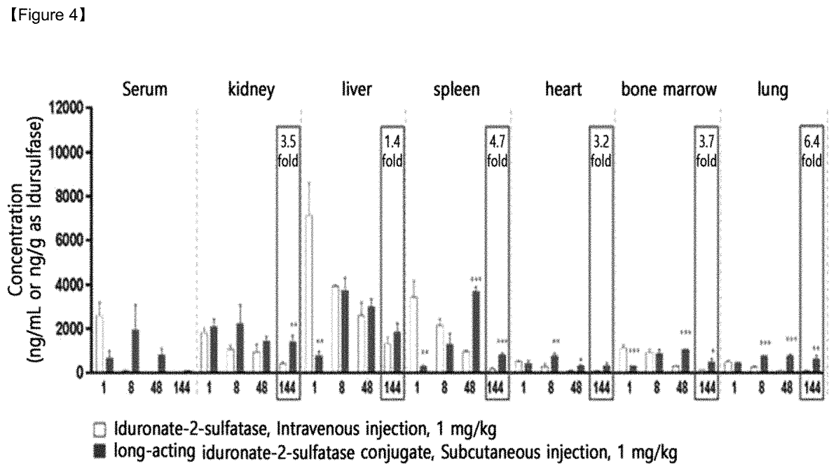

As a result, compared with the iduronate-2-sulfatase used as the control group, the long-acting iduronate-2-sulfatase conjugate showed high tissue distribution in all tissues at the same time (FIG. 4).

The above results suggest that the enzyme conjugates of the present invention are superior to other conventional therapeutic enzymes in the AUC and tissue distribution, and thus the enzyme conjugates can be used as epoch-making therapeutic agents for lysosomal storage diseases.

Those skilled in the art to which the present invention belongs will be able to understand that the present invention can be implemented in other detailed forms without changing the technical spirit or essential characteristics. Therefore, the embodiments described above are only examples and should not be construed as being limitative in all respects. The scope of the present invention is represented by claims to be described below rather than the detailed description, and it is to be interpreted that the meaning and scope of the claims and all of the changes or modified forms derived from the equivalents thereof come within the scope of the present invention.

1. An enzyme conjugate in which an immunoglobulin Fc region is linked to iduronate-2-sulfatase through a non-peptide polymer linker moiety.

2. The enzyme conjugate of claim 1, wherein the iduronate-2-sulfatase is able to treat mucopolysaccharidosis II (MPS II).

3. The enzyme conjugate of claim 1, wherein the enzyme conjugate has increased transcytosis and bioavailability (BA) as compared with iduronate-2-sulfatase to which the immunoglobulin Fc region is not linked.

4. The enzyme conjugate of claim 3, wherein the transcytosis is caused by binding of an immunoglobulin Fc region to a neonatal Fc receptor (FcRn).

5. The enzyme conjugate of claim 1, wherein the enzyme conjugate has increased tissue distribution as compared with iduronate-2-sulfatase to which the immunoglobulin Fc region is not linked.

6. The enzyme conjugate of claim 5, wherein the enzyme conjugate has increased bone marrow targeting as compared with iduronate-2-sulfatase to which the immunoglobulin Fc region is not linked.

7. The enzyme conjugate of claim 1,
wherein the immunoglobulin Fc region is aglycosylated;
wherein the immunoglobulin Fc region is constituted by 1 to 4 domains selected from the group consisting of CHL CH2, CH3, and CH4 domains;
wherein the immunoglobulin Fc region further comprises a hinge region;
wherein the immunoglobulin Fc region is an immunoglobulin Fc fragment derived from IgG, IgA, IgD, IgE, or IgM;
wherein each domain of the immunoglobulin Fc region is a hybrid of domains having different origins derived from an immunoglobulin selected from the group consisting of IgG, IgA, IgD, IgE, and IgM;
wherein the immunoglobulin Fc region is a dimer or multimer consisting of single-chain immunoglobulins composed of domains of the same origin; or
wherein the immunoglobulin Fc region is an IgG4 Fc fragment or wherein the immunoglobulin Fc region is a human aglycosylated IgG4 Fc fragment.

8-14. (canceled)

15. The enzyme conjugate of claim 1, wherein in the enzyme conjugate, a non-peptide polymer linker moiety is linked to a N-terminus of iduronate-2-sulfatase.

16. A composition comprising the enzyme conjugate of claim 1.

17. The composition of claim 16, wherein the composition increases transcytosis, bioavailability, tissue distribution, and bone marrow targeting.

18. A method for preparing an enzyme conjugate represented by the Chemical Formula 1 comprising:
(a) reacting any one reactive functional group of a non-peptide polymer in which the same or different reactive functional groups are positioned at both termini with free iduronate-2-sulfatase to obtain a linked material in which a non-peptide polymer is covalently linked to the iduronate-2-sulfatase through the terminus; and
(b) reacting a reactive functional group of an unreacted terminus of the linked material with a biocompatible substance capable of increasing a half-life in vivo to covalently link the biocompatible substance with the linked material,
X—La—F [Chemical Formula 1]
wherein, X is iduronate-2-sulfatase;
L is a non-peptide polymer linker moiety;
a is 0 or a natural number, but when a is 2 or more, each L is independent of one another; and
F is a substance capable of increasing the in vivo half-life of X.

19. The method for preparing an enzyme conjugate of claim 18, wherein the iduronate-2-sulfatase is able to treat mucopolysaccharidosis II (MPS II).

20. The method for preparing an enzyme conjugate of claim 18, wherein the F is selected from the group consisting of a polymer, a fatty acid, a cholesterol, albumin and fragments thereof, an albumin binding substance, a polymer of repeat units of a specific amino acid sequence, an antibody, antibody fragments, an FcRn binding substance, an in vivo connective tissue, a nucleotide, fibronectin, transferrin, saccharide, heparin, and elastin.

21. The method for preparing an enzyme conjugate of claim 20, wherein the FcRn binding substance is an immunoglobulin Fc region.

22. The method for preparing an enzyme conjugate of claim 18, wherein the non-peptide polymer is selected from the group consisting of polyethylene glycol, polypropylene glycol, an ethylene glycol-propylene glycol copolymer, polyoxyethylated polyol, polyvinyl alcohol, polysaccharide, dextran, polyvinyl ethyl ether, a biodegradable polymer, a lipid polymer, chitin, hyaluronic acid, and combinations thereof.

23. The method for preparing an enzyme conjugate of claim 22, wherein the non-peptide polymer is polyethylene glycol.

24. The method for preparing an enzyme conjugate of claim 18, wherein the reactive functional group is selected from the group consisting of an aldehyde group, a maleimide group, and a succinimide derivative.

25. The method for preparing an enzyme conjugate of claim 24, wherein the reactive functional group is one of the following (i)-(v):
(i) aldehyde group selected from the group consisting of a propionaldehyde group and a butyraldehyde group;
(ii) a succinimide derivative selected from the group consisting of succinimidyl carboxymethyl, succinimidyl valerate, succinimidyl methyl butanoate, succinimidyl methyl propionate, succinimidyl butanoate, succinimidyl propionate, N-hydroxysuccinimide, and succinimidyl carbonate;
(iii) aldehyde groups at both termini of the non-peptide polymer;
(iv) an aldehyde group at one terminus of the non-peptide polymer and a maleimide group at the other terminus; or
(v) an aldehyde group at one terminus of the non-peptide polymer and a succinimide group at the other terminus.

26-29. (canceled)

30. A method for preventing or treating mucopolysaccharidosis II (MPS II) comprising administering the enzyme conjugate of claim 1 to a subject in need thereof.
